(19) United States
(12) Patent Application Publication
(10) Pub. No.: US 2010/0233302 A1
(43) Pub. Date: Sep. 16, 2010

(54) THE USE OF A VEGF BINDING PEPTIDE TO RETARD HAIR GROWTH

(75) Inventors: Magali Bertrand, Chartres (FR); Farid Moussouni, Montvale, NJ (US); Anne Tindal, Hull (GB); Frederic DE Torre, 644 Chemin du Puy (FR)

Correspondence Address: TROUTMAN SANDERS LLP;5200 BANK OF AMERICA PLAZA, 600 PEACHTREE STREET, N.E., SUITE 5200, ATLANTA, GA, 30308-2216, US

(73) Assignee: Reckitt Benckiser (UK) Limited, Berkshire (GB)

(21) Appl. No.: 12/160,475

(22) PCT Filed: Jan. 26, 2007
(86) PCT No.: PCT/GB2007/000271

§ 371 Date: Feb. 18, 2010

(30) Foreign Application Priority Data
Jan. 27, 2006 (GB) 0601684.4

(51) Int. Cl. A61K 36/736; A61K 38/16; A61K 36/185; A61K 8/97; A61K 8/64; A61Q 7/02; A61P 17/00; A61Q 7/00

(52) U.S. Cl. 424/735; 514/12; 424/769

(57) ABSTRACT

Use of a peptide comprising a Bowman-Birk molecule in which the chymotrypsin inhibitory sequence and/or trypsin inhibitory sequence is replaced with a vascular endothelial growth factor binding sequence to retard hair growth.

This invention relates to the use a peptide for retarding hair growth.

Personal care compositions are known for removing unwanted hair from a desired location on the body of a user. Two general types of hair removing action are depilation and epilation. Depilation generally consists of utilising chemical means to dissolve or breakdown hair, thereby removing the hair from the desired body part. Epilation, on the other hand, generally consists of mechanically removing hair by plucking, cutting, waxing or the like, for example.

With both depilatory and epilatory personal care compositions, their use is to remove hair already present on the body of the user, with a view to leaving the skin on the part of the body to which the depilation or epilation is undertaken, free from hair and relatively smooth.

A particular problem with the use of depilatory and epilatory means to remove hair is that after application of the depilatory or epilatory means, and the removal of the hair, the hair grows back on the skin relatively quickly, and multiple applications of the depilatory or epilatory means must be performed frequently.

It would therefore be desirable to provide a personal care composition which induces hair growth retardation when applied to a desired body part of the user. It would also be desirable to provide a personal care composition which retards hair growth, with minimal side effects.

It is an aim of preferred embodiments of the present invention to provide a personal care composition which retards hair growth on a desired body part of a user.

Further, it is an aim of preferred embodiments of the present invention to overcome or mitigate at least one problem with the prior art.

According to a first aspect of the present invention, there is provided the use of a peptide comprising a Bowman-Birk molecule in which the chymotrypsin inhibitory sequence and/or trypsin inhibitory sequence is replaced with a vascular endothelial growth factor binding sequence (hereinafter designated BBI-AV molecule), to retard hair growth.

By “retarding” we mean inhibiting hair growth, as opposed to removal of existing hair as effected by depilation and epilation, for example.

According to a second aspect of the present invention, there is provided a hair anti-regrowth composition, not being a depilatory composition, which composition comprises a peptide comprising a Bowman-Birk molecule in which the chymotrypsin inhibitory sequence and/or trypsin inhibitory sequence is replaced with a vascular endothelial growth factor binding sequence; and a skin conditioning agent.

The Bowman-Birk protease inhibitor (BBI) is a designation of a family of stable, low molecular weight trypsin and chymotrypsin enzyme inhibitors found in soybeans and various other seeds, mainly leguminous seeds and vegetable materials. BBI comprises a family of disulfide bonded proteins with a molecular weight of about 8 kD (see e.g. Chou et al, Proc. Natl. Acad. Sci. USA 71:1748-1752 [1974]; Yavelow et al, Proc. Natl. Acad. Sci. USA 82:5395-5399 [1985]; and Yavelow et al., Cancer Res. (Suppl.) 43:2454s-2459s [1983]). BBI has a pseudo-symmetrical structure of two tricyclic domains each containing an independent native binding loop, the native loops containing binding sites for both trypsin and chymotrypsin (see Liener, in Summerfield and Bunding (eds), Advances in Legume Science, Royal Bot. Gardens, Kew, England). These binding sites each have, a canonical loop structure, which is a motif found in a variety of serine proteinase inhibitors (Bode and Huber, Eur. J. Biochem. 204:433-451 [1992]). Commonly, as in one of the soybean inhibitors, one of the native loops inhibits trypsin and the other inhibits chymotripsin (see Chen et al, J. Biol. Chem. 267:1990-1994 [1992]; Werner & Wemmer, Biochem. 212:549-555 [1993]; and Voss et al, Eur. J. Biochem. 242:122-131 [1996]) though in other organisms (e.g. Arabidopsis), both loops are specific for trypsin.

Suitably the BBI-AV molecule comprises a peptide having at leat 85% homology to the amino acid sequence of SEQ ID1. In preferred embodiments, the personal care composition includes a BBI-AV molecule having at least 90%, preferably 95%, more preferably 97% and most preferably 99%, and especially substantially 100% homology with SEQ ID1. The peptide comprising SEQ ID1 may be constructed according to the methods described in U.S. 2005/0203026 A1.

```
SEQ ID 1:
Asp Asp Glu Ser Ser Lys Pro Cys Cys Asp Gln Cys Ala Cys Thr Lys
1 5 10 15
Ser Asn Pro Pro Gln Cys Arg Cys Ser Asp Met Arg Leu Asn Ser Cys
                        20 25 30
His Ser Ala Cys Lys Ser Cys Ala Cys Tyr Asn Leu Tyr Gly Trp Thr
                35 40 45
Cys Phe Cys Val Asp Ile Thr Asp Phe Cys Tyr Glu Pro Cys Lys Pro
        50 55 60
Ser Glu Asp Asp Lys Glu Asn
65 70
```

SEQ ID1 comprises a peptide corresponding to the Bowman-Birk inhibitor molecule which has been engineered to replace the chymotrypsin inhibitory loop with a VEGF2 binding sequence (VEGF=vascular endothelial growth factor). Its construction is described in U.S. 2005/0203026 A1.

Preferably the BBI-AV molecule comprises the Bowman-Birk molecule in which the chymotrypsin inhibitory sequence has been replaced with a vascular endothelial growth factor binding sequence.

Preferably the BBI-AV molecule comprises an amino acid sequence corresponding to SEQ ID1.

Preferably the BBI-AV molecule is present in an amount of at least 0.005% wt, preferably at least 0.01% wt, preferably at least 0.02% wt, based on the total weight of the composition.

Preferably the BBI-AV molecule is present in the composition in an amount of up to 1% wt, preferably up to 0.5% wt, more preferably up to 0.2% wt, most preferably up to 0.1% wt, preferably up to 0.08% wt, preferably up to 0.05% wt, based on the total weight of the composition.

A preferred range for the BBI-AV is 0.01% wt to 0.08% wt, for example 0.02 to 0.05% wt, based on the total weight of the composition.

The skin conditioning agent of the present invention may suitably be selected from one or more skin healing agents, one or more skin penetration agents; or a mixture thereof.

A skin healing agent is a component which, for example, heals or sooths damaged or irritated skin. Skin healing agents suitable for use in the present invention include allantoin, sweet almond oil, witch hazel extract, and bisabolol.

Skin penetration agents are agents which assist a composition to penetrate the pores of skin. For example, following shaving, the pores in the skin may contract and this may make it difficult to topically deliver actives to the pores in the skin. In particular, it is advantageous if a hair retarding active agent can be delivered to the base of follicles. Ingress into pores is assisted by relaxation and/or opening of the pores.

Preferred penetration actives for use in the invention include ethanol, acetone, isopropyl alcohol and witch hazel extract. The use of such components may allow the amount of preservatives used in the composition to be reduced.

The skin conditioning agent or agents is/are preferably present in the composition in an amount of at least 0.05 wt %, preferably at least 0.1 wt %. Preferably the skin conditioning agent/s is/are present in an amount of up to 5 wt %, preferably up to 1 wt %, more preferably up to 0.5 wt %. This represents the total amount of all skin conditioning agents present in the composition.

Preferred skin healing agents include sweet almond oil and allantoin. In preferred embodiments, both of these components may be present. Allantoin may suitably be present in an amount from 0.05% wt to 0.5% wt, for example about 0.2% wt. Sweet almond oil may be present in an amount of from 0.05% wt to 0.5% wt, for example about 0.1% wt.

Suitably the hair anti-regrowth composition comprises, in addition to the BBI-AV molecule and skin conditioning agent, a physiologically acceptable carrier or excipient.

Preferably the carrier is at least one compound selected from the group consisting of water, an alcohol, (such as propylene glycol, ethanol, propenol, glycerol, butylene glycol and polyethylene glycol) or any mixture thereof.

Suitably the hair anti-regrowth composition comprises water as a carrier, more preferably in an amount of at least 50% wt of the total weight of the composition, still more preferably at least 65% wt of the total weight of the composition, and most preferably at least 75% wt of the total weight of the composition.

The hair anti-regrowth composition may be in the form of an emulsified vehicle, a cream or lotion, liposomal system, stabilised gel or dispersion system, a treatment serum, a topical pack or mask, a surfactant-based cleansing system such as a shampoo, body wash or body lotion, for example, an aerosolised or sprayed dispersion or emulsion, or a pigmented product such as make-up. In other preferred embodiments, the personal care composition may be a soap, shaving gel, shaving foam, deodorant aerosol, deodorant cream, deodorant stick or the like.

The hair anti-regrowth composition is preferably suitable for topical application to the skin or hair.

The hair anti-regrowth composition may be coated onto, and/or impregnated into a substrate to form a wipe comprising the personal care composition.

The substrate may be any suitable substrate such as paper, card, textiles, plastics or any mixture thereof for example. Textile substrates may comprise synthetic and/or natural textiles materials, including, but not limited to cotton, wool, rayon, urethanes, polyurethanes, polyesters, polyamides, rubber, nylon, hemp, jute, flax, leather, viscose, silk, and any mixture thereof.

The hair anti-regrowth composition may include further active ingredients in addition to the BBI-AV molecule and skin conditioning agent. Suitable active ingredients include, but are not limited to, vitamins and their derivatives, anti-oxidants, proteins and peptides, keratolytic agents, bio-flavinoids, terpenoids, phytochemicals, extracts of plant, marine or fermented origin. Preferably, such actives include, but are not limited to, antioxidants, such as tocopheryl and ascorborbyl derivatives, vitamins and vitamin derivatives, hydroxyl and polyhydroxy acids, enzymes and enzyme inhibitors, and any mixtures thereof.

The hair anti-regrowth composition may include a sunscreen, preferably having UVA-absorbing properties and/or UVB-absorbing properties. In some embodiments, the compositions of the present invention may include one or more UVA-absorbing sunscreen actives that absorb UV radiation having a wavelength of from about 320 nm to about 400 nm. Suitable UVA-absorbing sunscreen actives include, but are not limited to, dibenzoylmethane derivatives, anthranilate derivatives, and mixtures thereof. In some embodiments, the compositions of the present invention include one or more UVB sunscreen actives that absorb UV radiation having a wavelength of about 290 nm to 320 nm. Suitable UVB sunscreen actives include 2-ethylhexyl-2-cyano-3, 2-ethylhexyl-N,N-dimethyl-P-aminobenzoate, P-aminobenzoate acid, oxybenzone, octylsalicylate and the like, for example.

In some preferred embodiments, the hair anti-regrowth composition contains a whitener and/or an ultra-violet light blocking material, preferably in an amount of from 0.01% wt to 1% wt, preferably 0.05% wt to 0.4% wt, based on the total weight of the composition. Titanium dioxide is preferred. An advantage of the use of such a component is that lower levels of preservatives can sometimes be used.

The hair anti-regrowth compositions may include any other suitable ingredients, including one or more surfactants, natural oils, chelating agents (such as sodium EDTA), salts, organic acids (such as citric acid, for example), humectants, emulsifiers, colours, dyes, pigments, foaming agents, co-solvents, thickening agents, and any other suitable ingredients.

The hair anti-regrowth composition may include one or more surfactants selected from anionic, cationic, non-ionic or amphoteric (zwitterionic) surfactants.

Examples of anionic surfactants which may be used in the personal care composition include but are not limited to: alkali metal salts, ammonium salts, amine salts, aminoalcohol salts or the magnesium salts of one or more of the following compounds: alkyl sulphates, alkyl ether sulphates, alkylamidoether sulphates, alkylaryl polyether sulphates, monoglyceride sulphates, alkylsulphonates, alkylamide sulphonates, alkylarylsulphonates, olefinsulphonates, paraffin sulphonates, alkyl sulfosuccinates, alkyl ether sulfosuccinates, alkylamide sulfosuccinates, alkyl sulfosuccinamate, alkyl sulfoacetates, alkyl phosphates, alkyl ether phosphates, acyl sarcosinates, acyl isothionates and N-acyl taurates. Generally, the alkyl or acyl group in these various compounds comprises a carbon chain containing 12 to 30 carbon atoms.

Other anionic surfactants which may be used include fatty acid salts, including salts of oleic, ricinoleic, palmitic and stearic acids; copra oils or hydrogenated copra oil acid, and acyl lactylates whose acyl group contains 8 to 20 carbon atoms.

One class of nonionic surfactants which may be used in the personal care composition are alkoxylated alcohols, particularly alkoxylated fatty alcohols. These include ethoxylated and propoxylated fatty alcohols, as well as ethoxylated and propoxylated alkyl phenols, both having alkyl groups of from 5 to 16, more preferably 8 to 13 carbon chains in length.

Preferred nonionic surfactants are ethoxylated or propoxylated non-ionic surfactants prepared by the reaction of a monohydroxy alkanol or alkylphenol with 6 to carbon atoms with preferably at least 8 moles particularly preferred at least 10 moles, and still more preferred at least 15 moles of ethylene oxide or propylene oxide per mole of alcohol or alkylphenol.

Particularly preferred non-ionic surfactants are the non-ionics from a linear chain fatty alcohol with 12-20 carbon atoms ,and at least 8 moles particularly preferred at least 10 and still more preferred at least 15 moles of ethylene oxide or propylene oxide per mole of alcohol.

Preferred non-ionic surfactants additionally comprise propylene oxide units in the molecule. Preferably these PO units constitute up to 25% by weight, preferably up to 20% by weight and still more preferably up to 15% by weight of the overall molecular weight of the non-ionic surfactant. Particularly preferred surfactants are ethoxylated mono-hydroxy alkanols or alkylphenols, which additionally comprises polyoxyethylene-polyoxypropylene block copolymer units. The alcohol or alkylphenol portion of such surfactants constitutes more than 30%, preferably more than 50%, more preferably more than 70% by weight of the overall molecular weight of the non-ionic surfactant.

Another class of suitable non-ionic surfactants includes reverse block copolymers of polyoxyethylene and polyoxypropylene and block copolymers of polyoxyethylene and polyoxypropylene initiated with trimethylolpropane.

Another preferred class of nonionic surfactant can be described by the formula:

R¹O [CH₂CH (CH₃) O]ₓ [CH₂CH₂O]_{y} [CH₂CH (OH) R²]

where R¹ represents a linear or branched chain aliphatic hydrocarbon group with 4-18 carbon atoms or mixtures thereof, R² represents a linear or branched chain aliphatic hydrocarbon rest with 2-26 carbon atoms or mixtures thereof, x is a value between 0.5 and 1.5 and y is a value of at least 15.

Another group of preferred nonionic surfactants are the end-capped polyoxyalkylated non-ionics of formula:

R¹O [CH₂CH (R³)O]ₓ [CH₂] ₖCH (OH) [CH₂] ⱼOR²

where R¹ and R² represent linear or branched chain, saturated or unsaturated, alyphatic or aromatic hydrocarbon groups with 1-30 carbon atoms, R³ represents a hydrogen atom or a methyl, ethyl, n-propyl, iso-propyl, n-butyl, 2-butyl or 2-methyl-2-butyl group , x is a value between 1 and 30 and, k and j are values between 1 and 12, preferably between 1 and 5. When the value of x is >2 each R³ in the formula above can be different. R¹ and R² are preferably linear or branched chain, saturated or unsaturated, alyphatic or aromatic hydrocarbon groups with 6-22 carbon atoms, where group with 8 to 18 carbon atoms are particularly preferred. For the group R³ H, methyl or ethyl are particularly preferred. Particularly preferred values for x are comprised between 1 and 20, preferably between 6 and 15.

As described above, in case x>2, each R³ in the formula can be different. For instance, when x=3, the group R³ could be chosen to build ethylene oxide (R³=H) or propylene oxide (R³=methyl) units which can be used in every single order for instance (PO)(EO)(EO), (EO) (PO) (EO), (EO) (EO) (PO), (EO) (EO) (EO) , (PO) (EO) (PO), (PO) (PO) (EO) and (PO)(PO)(PO). The value 3 for x is only an example and bigger values can be chosen whereby a higher number of variations of (EO) or (PO) units would arise.

Particularly preferred end-capped polyoxyalkylated alcohols of the above formula are those where k=1 and j=1 originating molecules of simplified formula:

R¹O [CH₂CH (R³) O]ₓCH₂CH (OH) CH₂OR²

The use of mixtures of different nonionic surfactants is suitable in the context of the present invention for instance mixtures of alkoxylated alcohols and hydroxy group containing alkoxylated alcohols.

Examples of alkoxylated alcohols include certain ethoxylated alcohol compositions presently commercially available from the Shell Company, (Houston, Tex.) under the general trade name NEODOL (trade mark), which are described to be linear alcohol ethoxylates and certain compositions presently commercially available from the Union Carbide Company, (Danbury, Conn.) under the general trade name TERGITOL (trade mark), which are described to be secondary alcohol ethoxylates.

Examples of alkoxylated alkyl phenols include certain compositions presently commercially available from the Rhône-Poulenc Company (Cranbury, N.J.) under the general trade name IGEPAL (trade mark), which are described to be octyl and nonyl phenols.

Another class of non-ionic surfactants that may be used are sorbitan esters of fatty acids, typically of fatty acids having from 10 to 24 carbon atoms, for example sorbitan mono oleate.

Amphoteric surfactants which may be used in the present invention include amphoteric betaine surfactant compounds having the following general formula:

R_N⁺ (R₁) ₂_ R₂COO⁻

wherein R is a hydrophobic group which is an alkyl group containing from 10 to 22 carbon atoms, preferably from 12 to 18 carbon atoms, an alkylaryl or arylalkyl group containing a similar number of carbon atoms with a benzene ring being treated as equivalent to about 2 carbon atoms, and similar structures interrupted by amido or either linkages; each R₁ is an alkyl group containing from 1 to 3 carbon atoms; and R₂ is an alkylene group containing from 1 to 6 carbon atoms.

One or more such betaine compounds may be included in the personal care compositions used in the invention.

Examples of cationic surfactants which may be used in the personal care composition include quaternary ammonium compounds and salts thereof, including quaternary ammonium compounds which also have germicidal activity and which may be characterized by the general structural formula:

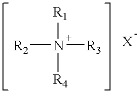

when at least one of R₁, R₂, R₃ and R₄ is a hydrophobic, aliphatic, aryl aliphatic or aliphatic aryl group containing from 6 to 26 carbon atoms, and the entire cationic portion of the molecule has a molecular weight of at least 165. The hydrophobic groups may be long-chain alkyl, long-chain alkoxy aryl, long-chain alkyl aryl, halogen-substituted long-chain alkyl aryl, long-chain alkyl phenoxy alkyl or aryl alkyl. The remaining groups on the nitrogen atoms, other than the hydrophobic radicals, are generally hydrocarbon groups usually containing a total of no more than 12 carbon atoms. The radicals R₁, R₂, R₃ and R₄ may be straight chain or may be branched, but are preferably straight chain, and may include one or more amide or ester linkages. The radical X may be any salt-forming anionic radical.

Examples of quaternary ammonium salts within the above description include the alkyl ammonium halides such as cetyl trimethyl ammonium bromide, alkyl aryl ammonium halides such as octadecyl dimethyl benzyl ammonium bromide, and N-alkyl pyridinium halides such as N-cetyl pyridinium bromide. Other suitable types of quaternary ammonium salts include those in which the molecule contains either amide or ester linkages, such as octyl phenoxy ethoxy ethyl dimethyl benzyl ammonium chloride and N-(laurylcocoaminoformylmethyl)-pyridinium chloride. Other effective types of quaternary ammonium compounds which are useful as germicides includes those in which the hydrophobic radical is characterized by a substituted aromatic nucleus as in the case of lauryloxyphenyltrimethyl ammonium chloride, cetylaminophenyltrimethyl ammonium methosulphate, dodecylphenyltrimethyl ammonium methosulphate, dodecylphenyltrimethyl ammonium chloride and chlorinated dodecylphenyltrimethyl ammonium chloride.

Preferred quaternary ammonium compounds which act as germicides and which are useful in the present invention include those which have the structural formula:

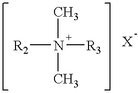

wherein R₂ and R₃ are the same or different C₈-C₁₂alkyl, or R₂ is C₁₂-C₁₆alkyl, C₈-C₁₈alkylethoxy, C₈-C₁₈alkyl-phenolethoxy and R₂ is benzyl, and X is a halide, for example chloride, bromide or iodide, or methosulphate. The alkyl groups R₂ and R₃ may be straight chain or branched, but are preferably substantially linear.

Other known surfactants not particularly described above may also be used. Such surfactants are described in McCutcheon's Detergents and Emulsifiers, North American Edition, 1982; Kirk-Othmer, Encyclopaedia of Chemical Technology, 3rd Ed., Vol. 22, pp 346-387. The present invention allows surfactants which are not thought to be compatible with other surfactants or other compounds to be used.

A composition of the present invention may comprise silicone oil. When present, this is preferably present in an amount of from 0.1% wt to 5% wt, preferably 0.5% wt to 3% wt, more preferably 1% wt to 2.5% wt, most preferably 1.5% wt to 2.5% wt.

The composition of the present invention preferably includes a buffer solution. The buffer solution suitably comprises deionised water, citric acid, sodium chloride and sodium hydroxide.

The pH of the composition is suitably maintained at between 5 and 7, preferably between 5.1 and 6.1.

According to a third aspect of thₑ invention, there is provided a cosmetic method of retarding hair growth comprising the steps of:
(a) providing a hair anti-regrowth composition as described for the first aspect of the invention; and
(b) contacting the composition with an area of skin in which it is desired to retard hair growth.

The method of the third aspect may suitably be carried out immediately following hair removal by, for example, depilation or epilation.

Preferably the composition is as described for the first and second aspects of the invention.

Depending on the form of the hair anti-regrowth composition, step (b) may comprise spraying, rubbing, massaging, jetting, kneading, washing or spreading ₜhe personal care composition onto the desired area of skin.

### EXAMPLES

In order for the various aspects of the invention to be more clearly understood, the invention will now be described by way of the following examples.

### Example 1

### Formulation A

A hair anti-regrowth composition containing a Bowman-Birk inhibitor molecule in which the chymotrypsin inhibitory sequence has been replaced by a vascular endothelial growth factor binding sequence (“BBI-AV molecule”), corresponding to the amino acid sequence of SEQ ID1, was prepared according to the following composition in Table 1.

TABLE 1
FORMULATION A - HAIR ANTI-REGROWTH FORMULATION
(LOTION) INCLUDING BBI-AV MOLECULE

| Ingredient (INCI name) | % in weight |
|---|---|
| Water | 85.97 |
| Cetearyl alcohol | 8.50 |
| Ceteareth-20 | 2.70 |
| PPG-15 Stearyl Ether | 0.60 |
| Prunus Amygdalus Dulcis Oil (1) | 0.10 |
| Paraffinum Liquidum | 0.10 |
| Allantoin | 0.20 |
| Propylene glycol | 0.13 |
| CI77891 Titanium dioxide | 0.17 |
| Tetra sodium EDTA | 0.10 |
| BBI-AV | 0.10 |
| Sodium Chloride | 0.091 |
| Citric acid | 0.11 |
| Sodium Hydroxyde | 0.034 |
| Phenoxyethanol | 1.0 |
| Robertet Artlande G10029876 (2) | 0.10 |

(1) Almond Oil supplied by Jan Decker
(2) Fragrance supplied by Robertet.

The formulation was manufactured by the following method:
1) Blending the fatty alcohol, emulsifier and oil gelling agent together into a molten phase at a temperature of 60, preferably 70° C. or more,
2) emulsifying the molten phase into an aqueous phase, the temperature of the aqueous phase prior to emulsification being 50° C., preferably 60° C., more preferably 70° C. or more, whereby an emulsion is formed,
3) cooling the emulsion to a temperature of 35° C. or less,
4) dispersing the perfume, preservative, citric acid solution buffer solution in the emulsion.
5) adding in the same manner the solution of BBi-Av, finishing with the buffer solution over a period of approximately 5 min.
6) Agitating the mixture for a further 10 minutes

### Control Formulation B

A control formulation, Formulation B, was manufactured in the same way as Formulation A, which included all of the ingredients of Formulation A set out in Table 1 above, except for the BBI-AV molecule.

A sample of volunteers (24 volunteers) then shaved an area of skin on one of their lower legs until no hair was visible on said area. Twelve of the volunteers then applied Formulation A of the invention to the shaved patch of skin, by applying the lotion into the desired area. At the same time, twelve of the volunteers applied the Control Formulation B to the shaved patch of skin by applying Control Formulation B into the desired area of skin.

Neither the volunteers nor the test supervisors were informed of the allocation of formulations A and B.

In addition, volunteers were asked:
not to remove hair from their lower legs or to use any other hair regrowth inhibitor product (on any body part).
not to use any cosmetic products (other than sun protection products) on their lower legs.
not use any type of artificial tanning products on their lower legs nor visit a tanning salon during the study.

Volunteers were asked to shave once every week.

Every day for eight weeks, the volunteers applied either Formulation A of the invention or Control Formulation B into the shaved patch of skin, as applicable, and the following characteristics were measured every week up to eight weeks, on the patch of skin to which Formulation A or Control Formulation B had been applied:

Mean hair count, mean hair length and mean hair diameter.

The results indicated that treatment with Formulation A of. the invention reduced mean hair count by approximately 24% compared to treatment with Control Formulation B after eight weeks.

The mean hair length of the hairs on the shaved patch of skin after eight weeks, of the twelve volunteers who had applied Formulation A of the invention was 19% lower than on the volunteers who had applied Control Formulation B.

The mean hair diameter of the hairs on the shaved patch of skin after eight weeks, of the twelve volunteers who had applied Formulation A of the invention was approximately 8% less than the mean hair diameter of hair re-growth on the patch of skin, after eight weeks, in the twelve volunteers who had applied Control Formulation B.

The results indicate that the use of a hair anti-regrowth composition which includes a Bowman-Birk inhibitor in which the chymotrypsin inhibitory sequence has been replaced by a vascular endothelial growth-binding sequence, significantly retards hair growth in each of hair numbers, hair length and hair diameter, compared to formulations which do not include said molecule.

### Example 2

A composition was prepared with formulation described as formulation C below:

### Formulation C

| | |
|---|---|
| Cetearyl Alcohol | 3.750 |
| Ceteareth-20 | 1.250 |
| Arlamol E | 0.400 |
| Sweet Almond Oil | 0.100 |
| Light Mineral Oil | 0.100 |
| Silicone Oil DC 246 | 2.000 |
| Deionised Water (part 1) | 79.850 |
| Allantoin | 0.200 |
| Na4 EDTA P | 0.100 |
| White Paste (TiO₂ + PPG) | 0.300 |
| Fragrance VMC/930/00 | 0.100 |
| Phenonip | 1.300 |
| Citric acid | 0.050 |
| Deionised Water (part 2) | 0.500 |
| Buffer Solution | 9.975 |
| BBI-AV Active | 0.025 |
| Total Formula | 100.000 |

### Made up of:

| TDS | | |
|---|---|---|
| code | Raw Material | % w/w [symbol] |
| 106402 | Deionised Water (part 3) | 9.79 [symbol] |
| 118162 | Citirc Acid | 0.06 [symbol] |
| N/A | Sodium Chloride | 0.09 [symbol] |
| N/A | Sodium Hydroxide | 0.03 [symbol] |

[symbol]

indicates data missing or illegible when filed

This formulation C was used as a base formulation which contained 0.025% wt BBI-AV active. Formulation C1 and C2 were prepared which were identical except that formulation C1 contained 0.05% wt BBI-AV active and C2 contained 0.1% wt BBI-AV active.

The three compositions were tested by panelists 50 panelists were supplied with formulation C 71 with formulation C1 and 74 with formulation C2.

The panelists were asked to assess whether they noticed any difference in hair regrowth over a period of 8 weeks, having used the product daily.

Overall, 50% of respondents perceived a hair regrowth inhibition effect; this could be perceived within two weeks of usage. Over 50% overall felt that hair regrowth was softer and finer. The results of questions answered by panellists are summarised in respect of each of the formulations of C, C1 and C2 in table 3.

| QUESTION | C | C1 | C2 |
|---|---|---|---|
| % whose overall | 84% | 66% | 65% |
| opinion of the | | | |
| product was good, | | | |
| very good or | | | |
| excellent | | | |
| % who found cream | 74% | 61% | 69% |
| provided long- | | | |
| lasting smoothness | | | |
| % who found | 74% | 53% | 52% |
| smoothness lasted | | | |
| twice as long as | | | |
| usual hair routine | | | |
| % who noticed | 58% | 47% | 59% |
| reduction in which | | | |
| hair grew back after | | | |
| 4 weeks | | | |
| % who noticed hair | 68% | 54% | 63% |
| growing back softer | | | |
| % who noticed hair | 68% | 45% | 59% |
| growing back finer | | | |
| % having to remove | 68% | 48% | 60% |
| hair less often | | | |
| % who considered | 64% | 50% | 65% |
| produce effective at | | | |
| delaying hair | | | |
| regrowth | | | |

Overall the results shown that the consumer appeal and satisfaction with respect to formulation C containing 0.025% wt BBI-AV active is at least as good as the consumer satisfaction with respect to formulations Cl and C2 comprising 0.05 and 0.1 wt % of the active. This result is surprising and beneficial. It allows a product to be developed containing a lower amount of active thus being more cost effective.

1. A method of inhibiting hair growth, the method comprising applying, to an area of skin in which it is desired to retard hair growth, a peptide comprising a Bowman-Birk molecule in which a chymotrypsin inhibitory sequence and/or a trypsin inhibitory sequence is replaced with a vascular endothelial growth factor binding sequence to retard hair growth.

2. The method claimed in claim 1 in which the peptide comprises at least 90% homology with SEQ ID1.

3. A hair anti-regrowth composition, not being a depilatory composition, which composition comprises a peptide comprising a Bowman-Birk molecule in which a chymotrypsin inhibitory sequence and/or a chymotrypsin inhibitory sequences is replaced by a vascular endothelial growth factor binding sequence, and skin conditioning agent.

4. The hair anti-regrowth composition according to claim 3 in which the peptide comprises at least 90% homology with SEQ ID1.

5. The hair anti-regrowth composition according to claim 3 which comprises from 0.01% wt to 0.1% wt of the BBI-AV molecule.

6. The composition according to claim 3 in which the skin conditioning agent comprises a skin healing agent selected from allantoin, sweet almond oil, witch hazel oil, bisabolol and mixtures thereof.

7. The composition according to claim 3 in which the skin conditioning agent comprises a penetration agent selected from ethanol, acetone, isoproponol and witch hazel.

8. The composition according to claim 3 which has a pH of between 5.1 and 6.1.

9. The cosmetic method of retarding hair growth comprising the steps of:
a) providing a hair anti-regrowth composition as defined in claim 3;
b) contacting the composition with an area of the skin in which it is desired to retard hair growth.

10. The composition as defined in claim 3 to provide one or more of the following benefits:
reduction in the rate of hair regrowth;
reduction in amount of hair regrowth;
finer hair regrowth;
longer lasting softer hair regrowth;
smoothness following epilation or depilation, and or
reduction in necessary frequency of hair removal.


SEQUENCE LISTING (1 sequence)

SEQ ID NO: 1 - protein, 71 aa, Artificial
  synthetic BBI-VEGF fusion
DDESSKPCCDQCACTKSNPPQCRCSDMRLNSCHSACKSCACYNLYGWTCFCVDITDFCYEPCKPSEDDKEN